(19) United States
(12) Patent Application Publication
(10) Pub. No.: US 2002/0168350 A1
(43) Pub. Date: Nov. 14, 2002

(54) Methods for treating disorders of neuronal deficiency with bone marrow-derived cells

(76) Inventors: Timothy R. Brazelton, Cupertino, CA (US); Helen M. Blau, Menlo Park, CA (US)

Correspondence Address: MORRISON & FOERSTER LLP, 755 PAGE MILL RD, PALO ALTO, CA, 94304-1018, US

(21) Appl. No.: 09/993,045

(22) Filed: Nov. 13, 2001

Related U.S. Application Data
(60) Provisional application No. 60/247,128, filed on Nov. 10, 2000.

(51) Int. Cl. A61K 48/00

(52) U.S. Cl. 424/93.21; 424/93.7

(57) ABSTRACT

The invention provides novel methods of treating neurological disorders which result in the loss of neurons (neuronal deficiencies). Bone marrow-derived cells are administered to individuals suffering from neuronal deficiencies. Administration of bone marrow-derived cells results in formation of new neurons in the nervous system, thereby replacing lost neurons. The methods of the invention may also be used for memory augmentation in memory impaired individuals.

### CROSS-REFERENCE TO RELATED APPLICATION

This application claims priority of U.S. Provisional Application No 60/247,128, filed Nov. 10, 2000 entitled “Methods for Treating Disorders of Neuronal Deficiency with Bone Marrow-Derived Cells” by the same inventors, the entire contents of which are hereby incorporated herein by reference as if fully set forth herein.

### STATEMENT OF RIGHTS TO INVENTIONS MADE UNDER FEDERALLY SPONSORED RESEARCH

The research leading to this invention was supported by grants nos. AG00259, HD 18179, AG09521, and CA59717 from the National Institutes of Health. The U.S. Government may have rights in this invention.

### TECHNICAL FIELD OF THE INVENTION

The invention relates generally to the treatment of neurological disorders, and more particularly to the treatment of neurological conditions characterized by a loss of neurons, damaged neurons, or neurons with inadequate or suboptimal function in the peripheral and/or central nervous system.

### BACKGROUND OF THE INVENTION

Adult bone marrow contains hematopoietic stem cells (HSC) which are capable of restoring the entire range of hematopoietic cells. Thus, bone marrow transplant (BMT) has been used extensively to rescue subjects with bone marrow failure due to myelotoxic chemotherapy/radiotherapy or congenital and/or genetic defects.

While the capacity of HSC to form all hematopoietic cells has been long known, it has been more recently discovered that stem cells present in adult bone marrow also give rise to additional cell types. Donor marrow-derived cells have been found in a variety of tissues. For example, donor marrow-derived liver oval cells have been identified (Peterson et al., 1999, Science, 284:1168-1170), and donor marrow-derived nuclei have been found integrated into skeletal muscle fibers (Gussoni et al., 1999, Nature 401:390-394). Donor marrow-derived cells expressing microglial and astrocytic markers have also been found in the brain following BMT (Eglitis et al., 1997, Proc. Natl. Acad. Sci. USA 94(8):4080-4085; Kennedy et al., 1997, Blood 90(3):986-993)

BMT has also been investigated as a treatment for a number of conditions that do not involve bone marrow failure. For example, enzyme supplementation in lysosomal storage disorders has been attempted by BMT (Krivit et al., 1991, Neuromuscular Disorders 1(6):449-454). Additionally, an ongoing trial in multiple sclerosis seeks to eliminate the autoimmune component by replacing the patient's immune system by allogeneic BMT.

A wide variety of neurological conditions are characterized by a loss of neurons, damaged neurons, or neurons with inadequate or suboptimal function. Some disorders, such as Parkinson's disease, involve loss of a particular type of neuron (dopaminergic neurons), while other disorders, such as stroke, involve the loss of neurons at a particular location (e.g., in an area of the brain supplied by an artery which is blocked during the stroke).

Some attempts have been made to replenish cells lost in such disorders. Implantation of fetal neurons has been attempted as a treatment for Parkinson's disease, and fetal cells have also been used to bridge spinal cord transections. However, cells suitable for such implantation are in extremely limited supply and, because of their fetal origin, ethical questions surround their harvest and use. Additionally, the administration of such cells (which must be directly administered into the brain) causes damage to the brain.

Accordingly, there is a need in the art for new methods for treating disorders involving loss of neurons.

### SUMMARY OF THE INVENTION

The invention provides new methods for the treatment of neurological conditions characterized by loss of neurons, damaged neurons, or neurons with inadequate or suboptimal function in the central and/or peripheral nervous system (“neuronal deficiencies”). This also includes conditions that may be treated by inducing neuronal loss followed by neuronal replacement. The inventors have found that bone marrow-derived cells are capable of entering the nervous system and forming new neurons. Accordingly, the invention provides methods of treating “neuronal deficiencies” associated with loss of neurons by administering bone marrow-derived cells and as well as the delivery of genetically engineered neuronal progenitors that may be used to treat “neuronal deficiencies.”

The invention provides methods for treating neuronal deficiencies by administering bone marrow-derived cells to an individual having a neuronal deficiency, thereby inducing formation of new neurons in the nervous system of the subject; and ameliorating at least one symptom of the neuronal deficiency. In certain embodiments, the neuronal deficiency arises from a disorder selected from the group consisting of abnormalities of the central autonomic systems, congenital disorders and disorders arising from teratogen exposure, demyelinating diseases, diseases of peripheral nerves, disorders of the hypothalamus and pituitary, disorders of movement, disorders of the spinal cord and vertebral column, epilepsy, hypoxia, increased intracranial pressure, infectious disease, neoplasia, neurodegenerative disorders, neuronal disorders associated with aging and senile dementia, nutritional disorders, perinatal neuropathologies, radiation damage, schizophrenia, single gene disorders, toxic disorders, trauma, vascular disease, and psychiatric disorders other than schizophrenia. The neuronal deficiencies treated by the invention exclude neuronal deficiencies arising from a disorder selected from the group consisting of a lysosomal or peroxisomal disorder, Zellweger's disease, human immunodeficiency virus (HIV) infection, multiple sclerosis (MS), leucodystrophies, adrenomyeloneuropathy, a metachromatic leucodystrophy (including globoid cell leucodystrophy, metachromatic leucodystrophies, and Sanfilipo's disease), sulphatide lipidosis, amyotrophic lateral sclerosis, amyotrophic lateral sclerosis with frontal lobe dementia, a bone marrow ablation treatment, lymphoma, metastases of tumors which do not arise in the nervous system, infantile acid maltase deficiency (Pompe's disease), Ceroid lipofuscinosis, a deficiency of GM2 gangliosidase, systemic lupus erythematosus, thrombophilia associated with antiphospholipid antibodies or polycythemia, and anemia including Sickle cell disease, beta-Thalassemia major, and other thalassemias.

The invention also provides methods for improving memory function in an individual with deficient memory function, by administering bone marrow-derived cells to an individual having deficient memory function, thereby inducing formation of new neurons in the nervous system of the subject; and improving at least one memory function in the individual.

Preferably, the bone marrow-derived cells are autologous, syngeneic, or allogeneic, and the bone-marrow derived cells may be genetically modified. The use of xenogeneic cells is contemplated, but xenogeneic cells are less preferred. The cells may be administered by any method, such as by vascular administration (e.g., intravenously), intrathecally, or locally.

In some embodiments, the bone marrow-derived cells are administered in conjunction with a neuronal factor such as nerve growth factor (NGF), brain-derived neurotrophic factor (BDNF), neurotrophin-3, -4, -5, -4/5 and -6 (NT-3, -4, -5, -4/5, -6), ciliary neurotrophic factor (CNTF), glial-derived neurotrophic factor (GDNF), growth promoting activity (GPA), luteinizing hormone releasing hormone (LHRH), KAL gene (implicated in X-linked Kallman's syndrome), insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF-1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-β1, -β2, -β3, -β4, and -β5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies).

Also provided are methods for treating a neuron deficiency by administering a bone marrow cell mobilization therapy to an individual having a neuron deficiency, thereby inducing formation of new neurons in the nervous system of the subject; and ameliorating at least one symptom of the neuron deficiency.

Further embodiments of the invention provide for treating a neuron deficiency or for improving memory function by administering bone marrow-derived cells in combination with a bone marrow cell mobilization therapy to an individual having a neuron or memory deficiency, thereby inducing formation of new neurons in the nervous system of the subject; and improving symptoms of the neuronal deficiency or improving memory function in the individual.

### DESCRIPTION OF THE INVENTION

The inventors have found that administration of cells contained in the bone marrow results in formation of new neurons in the central nervous system. These cells do not express astrocytic, microglial, nor hematopoietic markers. Formation of neurons by bone marrow-derived cells has not, to the inventors' knowledge, been previously described.

The invention provides methods of treating neurological indications which involve the loss of neurons, damaged neurons, or neurons with inadequate or suboptimal function in the central and/or peripheral nervous system (“neuron deficiencies”) by administering bone marrow-derived cells. Administration of bone marrow-derived cells results in improvement in one or more symptoms of the neuron deficiencies being treated. Bone marrow-derived cells are already extensively used in clinical practice, although for other indications, and provide the advantage of being readily accessible (e.g., compared to fetal brain cells).

Additionally, the invention provides methods for increasing central and/or peripheral nervous system neurons or changing their function and/or connections (i.e., plasticity) by administering bone marrow-derived cells. These methods are useful for improving and/or stabilizing mental function, such as memory, particularly short term memory function, for correcting dysfunctions such as epilepsy, ataxias, and psychological disorders such as disorders of mood and/or affect, or for delivering genes to the CNS.

Also provided are methods for treating symptoms of a neuronal deficiency by administering a bone marrow cell mobilization therapy to a subject having a neuronal deficiency. Mobilization of bone marrow cells results in the formation of new neurons in the nervous system of the subject and further results in improvement in one or more symptoms of the neuronal deficiencies being treated.

Definitions

As used herein, the term “subject” or “individual” refers to a vertebrate, and includes avians and mammals. The term “mammal” refers to any individual of a mammalian species, and includes large animals (cows, sheep, horses and the like), sport animals (including dogs and cats), and primates (including old world monkeys, new world monkeys, apes, humans, and the like).

As used herein, the term “treating” refers to ameliorating, improving, reducing, or stabilizing one or more symptoms of a disorder or undesired condition, as well as slowing progression of one or more symptoms of a neuronal deficiency.

The term “neuronal deficiency”, as used herein, refers to a neurological disorder characterized by the actual or potential loss of neurons, damaged neurons, or neurons with inadequate or suboptimal function in the central and/or peripheral nervous system. Neuronal deficiencies include neurological disorders arising from disorders including abnormalities of the central autonomic systems, congenital disorders and disorders arising from teratogen exposure, demyelinating diseases, diseases of peripheral nerves, disorders of the hypothalamus and pituitary, disorders of memory or suboptimal memory, disorders of movement, disorders of the spinal cord and vertebral column, epilepsy, hypoxia, increased intracranial pressure, infectious disease, neoplasia, neurodegenerative disorders, neuronal disorders associated with aging and senile dementia, nutritional and metabolic disorders, perinatal neuropathologies, radiation damage, schizophrenia, psychiatric disorders other than schizophrenia, single gene disorders, toxic disorders, trauma, and vascular disease. As used herein, the term “neuronal deficiencies” excludes a number of diseases/disorders/syndromes: lysosomal and peroxisomal disorders, Zellweger's disease, neuronal deficiencies arising from human immunodeficiency virus (HIV) infection, multiple sclerosis (MS), adrenoleucodystrophy, adrenomyeloneuropathy, metachromatic leucodystrophies, sulphatide lipidoses, globoid cell leucodystrophy (Krabbe's disease, galactosylceramide lipidosis), amyotrophic lateral sclerosis, sporadic amyotrophic lateral sclerosis, amyotrophic lateral sclerosis with frontal lobe dementia, familial amyotrophic lateral sclerosis, and familial amyotrophic lateral sclerosis with frontal lobe dementia, bone marrow ablation (e.g., by chemotherapy), lymphomas (e.g., primary malignant lymphomas, secondary lymphomas, and Plasma cell tumors) as well as metastases of tumors which do not arise in the nervous system, infantile acid maltase deficiency (Pompe's disease), metachromatic leucodystrophy, Ceroid lipofuscinosis, deficiencies of GM2 gangliosidases, Sanfilipo's disease, leucodystrophy, systemic lupus erythematosus, thrombophilia associated with antiphospholipid antibodies or polycythemia, anemias (including Sickle cell disease, beta-Thalassemia major, and other thalassemias).

Neuronal deficiencies are listed below and organized into groups based on type of disease or location. However, as many disorders have complex outcomes and involve multiple sites and pathologies within the nervous system, it should be noted that the inventors contemplate that the disorders listed below should not be limited to the pathology or location of the group in which they are listed, but include all nervous system locations and pathologies affected by the named disorder.

Abnormalities of the central autonomic systems

The term “abnormalities of the central autonomic systems”, as used herein, refers to those neuronal deficiencies involving a loss of neurons, damaged neurons, or neurons with inadequate or suboptimal function due to central autonomic failure, including primary and secondary causes of autonomic failure such as progressive autonomic failure (including progressive autonomic failure with Lewy bodies, with multiple system atrophy, and due to postganglionic pathology), dopamine beta-hydroxylase deficiency, structural lesions of the spinal cord, brain stem, corticolimbic or hypothalamic regions, cerebrovascular disease, botulism, acute autonomic neuropathy, and peripheral neuropathies such as diabetic, amyloid, inflammatory, alcoholic, toxic, drug-related, chronic renal failure, paraneoplastic, connective tissue disease, acute intermittent porphyria, and familial neuropathy. The term “abnormalities of the central autonomic systems” excludes neuropathies related to lymphoreticular proliferative disorders (including lymphoma, leukemia, myeloma, and polycythemia vera).

Congenital disorders and teratogens

The term “congenital disorders and teratogens” refers to those neuronal deficiencies involving a loss of neurons, damaged neurons, or neurons with inadequate or suboptimal function associated with spinal cord malformations (including secondary degenerations), syringomyelia, syringobulbia, meningocele, spina bifida occulta, hydromyelia, diplomyelia, diastematomyelia, anomalies of the septum pellucidum (including secondary destructions), neuronal migration defects (e.g., laminar neuronal hetertopias, microdysgenesis, and hippocampal anomalies), encephaloclastic defects, megalencephaly, malformations/hypoplasias/dysplasias/atrophies of the cerebrum and cerebellum, pontoneocerebellar hypoplasia, granular layer aplasia, olivopontocerebellar atrophy in association with carbohydrate deficient glycoprotein (CDG) deficiency (disialotransferrin developmental deficiency syndrome), crossed cerebellar atrophy, cerebellar heterotopias, cerebellar cortical dysplasia, brain stem malformations, olivary dysplasia, dentate dysplasia, dentato-olivary dysplasia, arthogryposis multiplex congenta (AMC) syndrome (it should be noted that the term “AMC”, as used herein, excludes AMC associated with Zellweger's syndrome), malformations involving the nervous system associated with trisomy 21, trisomy 13, trisomy 14, trisomy 18, trisomy 8 mosaicism, fragile X syndrome, Lhermitte-Duclos disease, Dandy-Walker syndrome, Joubert's syndrome, septo-optic dysplasia, Fukuyama congenital muscular dystrophy, Walker-Warburg syndrome, cerebro-ocular dysplasia-muscular dystrophy syndrome (COD-MD), Möbius syndrome, Sturge-Weber syndrome, arachnoid cysts, phakomatosis, tuberous sclerosis, Bourneville's disease, hypomelanosis of Ito, Von Recklinghausen's disease, hydrocephalus, fetal alcohol syndrome, maternal phenylketonuria, maternal diabetes mellitus, and maternal infection with teratogenic infectious agents (such as rubella, cytomegalovirus, Herpes simplex, Herpes zoster, toxoplasmosis, and the like), vascular malformations involving the CNS and/or PNS, and maternal exposure to teratogens such as alcohol, carbamazepine, hyperthermia, methyl mercury, phenytoin, retinoids, valproic acid, varicella, warfarin, and X-irradiation.

Demyelinating diseases

The term “demyelinating disease” refers to neuron deficiencies involving a loss of neurons, damaged neurons, or neurons with inadequate function associated with demyelination due to viral causes such as progressive multifocal leucoencephalopathy, subacute sclerosing panencephalitis, canine distemper encephalomyelitis, mouse hepatitis virus (JHM) encephalomyelitis, Theiler's murine virus encephalomyelitis, Semliki Forest virus encephalomyelitis, Visna, Herpes simplex virus type I and type II infections, and Human T lymphotrophic virus Type I (HTLV I) associated myelopathy (tropical spastic paraplegia), phenylketonuria, autoimmune (or suspected autoimmune) causes including perivenous encephalomyelitis (postinfectious encephalomyelitis, postvaccinal encephalomyelitis), rabies postvaccinal encephalomyelitis, acute hemorrhagic leucoencephalitis (Hurst's disease), nutritional/metabolic causes which include disorders such as Marchiafava-Bignami disease, vitamin B12 deficiency (subacute combined degeneration), and central pontine myelinosis, toxic causes including hexachlorophene intoxication, and periventricular leucoencephalopathy associated with combined anti-mitotic medication and radiotherapy, and other causes which include disorders such as prolonged cerebral edema, hypoxic-ischemic leucoencephalopathy (carbon monoxide poisoning, anoxic and ischemic anoxia), and cerebrospinal fluid exchange, perivenous encephalomyelitis which includes acute disseminated encephalomyelitis, postinfectious encephalomyelitis, postvaccinal encephalomyelitis, and acute perivascular myelinoclasis, rabies postvaccinal encephalomyelitis, acute hemorrhagic leucoencephalitis (Hurst's disease), acquired hypomyelinogenesis congenita, starvation, protein deprivation, essential fatty acid deficiency, copper deficiency, vitamin B12 deficiency, electrolyte-induced demyelination, spinal cord compression, cerebrospinal fluid exchange, and X-irradiation of the CNS in young and mature animals. As used herein, a “demyelinating disease due to a viral cause” excludes human immunodeficiency virus (HIV) encephalopathy and HIV vacuolar myelopathy. “Demyelinating disease due to an autoimmune cause” excludes multiple sclerosis (MS), including variants of the disease. The term “demyelinating disease due to a genetic cause” excludes adrenoleucodystrophy, adrenomyeloneuropathy, metachromatic leucodystrophies, sulphatide lipidoses, and globoid cell leucodystrophy (Krabbe's disease, galactosylceramide lipidosis), neuropathies related to lymphoreticular proliferative disorders (including lymphoma, leukemia, myeloma, and polycythemia vera and hereditary neuropathies affecting peripheral nerves exclude metachromatic leucodystrophy (sulphatide lipidosis)), adrenoleucodystrophy, adrenomyeloneuropathy, G M₁ gangliosidosis, G M₂ gangliosidosis, Gaucher's disease, Niemann-Pick disease (including type A (type I) and type C (type II), Fabry's disease (angiokeratoma corporis diffusum), Wolman's disease, and Batten's disease.

Diseases of peripheral nerves

The term “diseases of peripheral nerves”, as used herein, refers to those neuronal deficiencies involving a loss of neurons, damaged neurons, or neurons with inadequate or suboptimal function due to neuronal and/or axonal degeneration/dysfunction including that associated with trauma, crush injury, stretch injury, transection, radiation neuropathy, distal axonopathy, ischemic injury, chronic nerve compression, cold injury, polyglucosan bodies, Strachan's syndrome, alcoholic neuropathy, peripheral neuropathies due to toxic neuropathies (including those due to environmental agents and biological agents such as including acrylamide, buckthorn, carbon disulphide, carbon monoxide, dimethylaminopropionitrile (DMAPN), diptheria toxin (including diphtheritic neuropathy), ethylene oxide, hexacarbons, metals (including arsenic, lead, mercury, and thallium), organophosphorus esters, drug-induced neuropathies (including those associated with colchicine, gold, isoniazid nucleosides (dideoxycytidine (ddC), dideoxyinosine (ddI), and stavudine (d4T)), platinum, taxol, and vincristine), neuropathies related to system metabolic disorders (e.g., uremic neuropathy and those associated with diabetes mellitus, hypoglycemia, and hypothyroidism) and amyloid neuropathies (including those associated with primary amyloidosis). The term “disorders of peripheral nerves” excludes neuropathies associated with HIV infection and neuropathies related to lymphoreticular proliferative disorders (including lymphoma, leukemia, myeloma, and polycythemia vera and hereditary neuropathies affecting peripheral nerves exclude metachromatic leucodystrophy (sulphatide lipidosis), globoid cell leucodystrophy (Krabbe's disease, galactosylceramide lipidosis), Refsum's disease, adrenoleucodystrophy, adrenomyeloneuropathy, G M₁ gangliosidosis, G M₂ gangliosidosis, Gaucher's disease, Niemann-Pick disease (including type A (type I) and type C (type II), Fabry's disease (angiokeratoma corporis diffusum), Farber's disease, Wolman's disease, amyloidosis associated with myeloma Waldenstrom's macroglobulinemia, and Batten's disease.

Disorders of the hypothalamus and pituitary

The term “disorders of the hypothalamus and pituitary”, as used herein, refers to those neuronal deficiencies involving a loss of neurons, damaged neurons, or neurons with inadequate or suboptimal function due to disorders of the hypothalamus and pituitary such as hypothalamic and posterior pituitary hyperfunction, hypothalamic and posterior pituitary hypofunction, malformations and hamartomas of the pituitary and hypothalamus, inflammatory lesions, infectious diseases, metabolic disorders, degenerative diseases, and vascular diseases.

Disorders of movement

The term “disorders of movement”, as used herein, refers to neuronal deficiencies involving a loss of neurons, damaged neurons, or neurons with inadequate or suboptimal function due to akinetic rigid movement disorders, Parkinsonism including Parkinson's disease, idiopathic Parkinson's disease, drug-induced parkinsonism, vascular pseudoparkinsonism, arteriosclerotic pseudoparkinsonism, Alzheimer-type changes, frontotemporal neurodegenerative disorders, juvenile parkinsonism, toxin-related parkinsonism, Guam parkinsonism, parkinsonism dementia complex of Guam, and postencephalitic parkinsonism, conditions characterized by abnormal stiffness such as stiff man syndrome, progressive encephalomyelitis with rigidity and Isaac's syndrome, hyperkinetic movement disorders including Huntington's disease, metabolic derangements, drug-induced chorea, and focal lesion-induced chorea, myoclonal disorders such as Creutzfeldt-Jakob disease, Lewy body disease, and Alzheimer's disease, dystonias, tic disorders including Gilles de la Tourette syndrome, ataxic disorders including Friedreich's ataxia, ataxia-telengiectasia, autosomal dominant cerebellar ataxias, episodic ataxias, and Wolfram's syndrome, motor neuron disorders including motor neuron disorders secondary to an infectious disease or toxin exposure (e.g., post-polio syndrome, and syphilis infection) and spinal muscular atrophies. The term “movement disorders” also includes disorders affecting basal ganglia including thalamic lesions (e.g., as occurs in Friedreich's ataxia, fatal familial insomnia, and in isolated thalamic degeneration) pallidal degenerations (e.g. as occurs in pure pallidal degeneration, pallidoluysial degeneration, pallidonigral degeneration, and pallidonigroluysial degeneration), neuroaxonal dystrophy and related disorders (including physiological neuroaxonal dystrophy, primary neuroaxonal dystrophies and secondary neuroaxonal dystrophies), disorders associated with mineralization of basal ganglia (including hypoparathyroidism, familial psychosis, pupus cerebritis, and folate deficiency, but excluding carbonic anhydrase II deficiency) disorders associated with calcification of basal ganglia (striatopallidodentate calcification, brain calcinosis, Fahr's disease), striatal necrosis (including that associated with hypoxia, hypoglycemia, carbon monoxide poisoning, and the like) and neuroleptic malignant syndrome (including the hyperthermia and multiorgan failure associated with 3,4-methylenedioxymethamphetamine (MDMA, ‘Ecstasy’). The term “disorders of movement”, particularly “hyperkinetic movement disorders”, excludes Batten's disease and systemic lupus erythematosus. Additionally, the term “disorders of movement”, particularly “motor neuron disorders”, excludes amyotrophic lateral sclerosis, sporadic amyotrophic lateral sclerosis, amyotrophic lateral sclerosis with frontal lobe dementia, familial amyotrophic lateral sclerosis, and familial amyotrophic lateral sclerosis with frontal lobe dementia, and HIV infection.

Disorders of the spinal cord and vertebral column

The term “disorders of the spinal cord and vertebral column,” as used herein, refers to those neuronal deficiencies involving a loss of neurons, damaged neurons, or neurons with inadequate or suboptimal function due to a disorder of the spinal cord and/or vertebral column, including vascular diseases such as occlusive vascular disease: (e.g., resulting in ischemic myelopathy), compression of spinal cord, diseases of the vertebral column affecting the spinal cord including intervertebral disc release and spondylosis, bony abnormalities in the region of the foramen magnum, rheumatoid arthritis and ankylosing spondylitis, spinal cord compression, infectious diseases involving vertebrae and meninges, neoplastic processes involving vertebrae and meninges, trauma including penetrating injuries, non-penetrating injuries, post-traumatic syringomyelia, partial or total spinal cord transection, and chronic adhesive spinal arachnoiditis. The term “disorders of the spinal cord and vertebral column “excludes neuropathies related to lymphoreticular proliferative disorders (including lymphoma, leukemia, myeloma, and polycythemia vera).

Epilepsy

The term “epilepsy” refers to those neuronal deficiencies characterized by chronic, recurrent paroxysmal changes in neurological function. Each episode is referred to as a “seizure”, and may present with motor, sensory, autonomic, or psychic symptoms. Seizures with motor symptoms are “convulsive” seizures”. Epilepsy includes status epilepticus, chronic loss of neurons, reactive gliosis, and iatrogenic damage relating to surgical or medical treatment. The term “epilepsy” includes idiopathic epilepsy, primary epilepsies, age-related onset epilepsies, childhood epilepsies, epilepsies secondary to other disorders, such as malformations, infantile Huntington's disease, vascular malformation(s), infection(s) and infectious diseases such as meningitis, encephalitis, parasite infection, and malaria, post-traumatic epilepsy, gliotic scar associated epilepsy, and epilepsies associated with intracranial tumors, infarcts, febrile episodes, and the like. As used herein, the term “epilepsy” excludes epilepsy associated with juvenile Gaucher disease, neuropathies related to lymphoreticular proliferative disorders (including lymphoma, leukemia, myeloma, and polycythemia vera), and Krabbe's disease.

Hypoxia

The term “hypoxia”, as used herein, refers to those neuronal deficiencies involving a loss of neurons, damaged neurons, or neurons with inadequate or suboptimal function due to hypoxias including hypoxic hypoxia, anemic hypoxia, stagnant hypoxia (including cardiac arrest encephalopathy and transient global ischemia), non-perfused brain (including respirator brain and permanent global ischemia), and histotoxic hypoxia) (and including hypoxia associated with carbon monoxide poisoning, air embolism, vascular disruption/blockage (including stroke and embolism) and decompression sickness. “Hypoxia” of the central nervous system, and particularly the brain, results in ischemic lesions. In certain embodiments, individual ischemic lesions in the CNS of a subject having a hypoxic neuron deficiency are less than about 5%, 2.5%, or 1% of total brain volume, although individual lesions may “fuse” to form aggregate lesions which are greater than 5%, 2.5%, or 1% of total brain volume (aggregate lesions can be recognized by the shape of their aggregated borders). As used herein, the term “hypoxia” excludes hypoxia related to lymphoreticular proliferative disorders (including lymphoma, leukemia, myeloma, and polycythemia vera).

Increased intracranial pressure

The term “increased intracranial pressure” refers to those neuronal deficiencies involving a loss of neurons, damaged neurons, or neurons with inadequate or suboptimal function due to raised intracranial pressure. Increased intracranial pressure may be due to a variety of causes, including changes in cerebrospinal fluid production or absorption or intracranial blood volume, brain swelling and edema, intracranial expanding lesions (including hemorrhage, hydrocephalus (including obstructive (non-communicating) hydrocephalus), and benign intracranial hypertension.

Infectious disease

The term “infectious disease” refers to those neuronal deficiencies involving a loss of neurons, damaged neurons, or neurons with inadequate or suboptimal function due to an infectious disease such as a viral infection (e.g., herpes virus infection, poliovirus infection (e.g. poliovirus acute encephalomyelitis), arbovirus infection (e.g., acute encephalitis caused by arboviruses), mumps, measles, rubella and the like), parasitic infections such as protozoal infections (e.g., amoebiasis, cerebral malaria, toxoplasmosis, and the like), fungal infections including Aspergillosis, Candidiasis, and the like, bacterial infections including pyogenic infections (e.g., abscess), mycoplasma infections such as Sarcoidosis, and prion diseases including scrapie. The term “infectious disease”, as used herein, excludes HIV infections (AIDS) as well as infections occuring in individuals with HIV infection (e.g., Aspergillosis or Candidiasis).

Lysosomal storage disorders

As used herein, the term “lysosomal storage disorder” refers to an inborn error of metabolism which results in a build up of one or more substances in the lysosomal compartment of cells of an individual afflicted with the disorder. Lysosomal storage disorders may result in mental and/or physical disabilities and may additionally reduce the life expectancy of the afflicted individual, depending on the identity and severity of the particular lysosomal storage disorder. The known lysosomal storage disorders include: Pompe's disease (acid-a1,4-glucosidase deficiency), G_{M1},-gangliosidosis including the infantile form (type 1), pseudo-Hurler disease, Tay-Sachs with visceral involvement, familial neurovisceral lipidosis, Landing's disease, generalized gangliosidosis, and adult G_{M1}-gangliosidosis (type 3), Tay-Sachs disease (β-hexosaminidase A deficiency), G_{M2}-gangliosidosis including the infantile (Tay-Sachs) forms (types B, O and AB) and the infantile, late infantile, juvenile or adult forms (types B and B1), G_{M3}-gangliosidosis, G_{D3}-gangliosidosis, Sandhoff disease (β-hexosaminidase A & B deficiency), Fabry disease (α-galactosidase A deficiency), Gaucher disease (glucocerebrosidase deficiency) including Type 1 (chronic non-neuronopathic Gaucher's disease), Type 2 (acute neuronopathic Gaucher's disease), Type 3 (the Norrbottnian type, or subacute or juvenile neuronopathic Gaucher's disease), types A and B Nieman-Pick (acid sphingomyelinase deficiency), type C Nieman-Pick (cholesterol esterification defect), type D Nieman-Pick, Farber disease (acid ceramidase deficiency), Wolman's disease (acid lipase deficiency), mucopolysccharidosis (MPS) IH (Hurler's syndrome/disease, α-L-iduronidase deficiency), MPS IS (Scheie syndrome/disease, α-L-iduronidase deficiency), MPS IH/S (Hurler-Scheie syndrome/disease, α-L-iduronidase deficiency), MPS II (Hunter's syndrome/disease, iduronate sulfatase deficiency), MPS II subtype A (severe Hunter's syndrome/disease), MPS II subtype B (mild Hunter's syndrome/disease), MPS III (Sanfilippo's syndrome/disease), MPS III subtype A (subtype A Sanfilippo's syndrome/disease, heparan N-sulfatase deficiency), MPS III subtype B (subtype B Sanfilippo's syndrome/disease, α-N-acetylglucosaminidase deficiency), MPS III subtype C (subtype C Sanfilippo's syndrome/disease, acetyl-CoA-glucosaminide acetyltransferase deficiency), MPS III subtype D (subtype D Sanfilippo's syndrome/disease, N-acetylglucosamine-6-sulfatase deficiency), MPS IV (Morquio's syndrome/disease), MPS IV subtype A (galactosamine-6-sulfatase deficiency aka Morquio A or severe Morquio's syndrome/disease), MPS IV subtype B (Morquio B/mild Morquio's syndrome/disease, β-galactosidase deficiency), MPS VI (Maroteaux-Lamy's syndrome/disease, arylsulfatase B deficiency), MPS VI subtype A (severe Maroteaux-Lamy's syndrome/disease), MPS VI subtype B (mild Maroteaux-Lamy's syndrome/disease), MPS VII (Sly's syndrome/disease, β-glucuronidase deficiency), mannosidoses including mannosidosis, alpha-mannosidosis, severe infantile alpha-mannosidosis, severe infantile type I alpha-mannosidosis infantile alpha-mannosidosis, type I alpha-mannosidosis, juvenile-adult alpha-mannosidosis, type II alpha-mannosidosis, mild alpha-mannosidosis, juvenile-adult type II alpha-mannosidosis, beta-mannosidosis, and other variants, fucosidosis (α-L-fucosidase deficiency) including type I fucosidosis, infantile fucosidosis, and type II fucosidosis, aspartylglucosaminuria (N-aspartyl-β- glucosaminidase), sialidosis (α-neuraminadase deficiency, aka mucolipidosis I), galactosialidosis (lysosomal protective protein deficiency, aka Goldberg syndrome), Schindler disease (α-N-acetyl-galactosaminidase deficiency), mucolipidosis II (N-acetylglucosamine-1-phosphotransferase deficiency, aka I-cell disease), mucolipidosis III (N-acetylglucosamine-1-phosphotransferase deficiency, aka pseudo-Hurler polydystrophy), cystinosis (cystine transport protein deficiency) including severe neuropathic cystinosis, infantile cystinosis, intermediate cystinosis, childhood cystinosis, juvenile cystinosis, adult cystinosis, and benign cystinosis, sialurias including sialuria, Salla disease (sialic acid transport protein deficiency), and infantile sialic acid storage disease (sialic acid transport protein deficiency), infantile neuronal ceroid lipofuscinosis (palmitoyl-protein thioesterase deficiency), mucolipidosis IV, and prosaposin (saposin A, B, C or D deficiency), G_{M1}-gangliosidosis including the infantile form (type 1), Batten's disease including neuronal ceroid lipofuscinosis (NCL), Bielschowsky-Jansky disease, Spielmeyer-Vogt-Sjögren disease, Stengel's disease, amaurotic familial idiocy, cerebral lipidosis with onset past infancy, cerebromacular degeneration, diffuse lipofuscinosis, heredofamilial lipidosis, maculocerebral degeneration, neurovisceral storage disease with curvilinear bodies, polyunsaturated fatty acid lipidosis, infantile Batten's disease, (CLN1), Late-infantile Batten's disease (CLN2), Juvenile Batten's disease (CLN3), Adult Batten's disease (CLN4), Kufs' disease, Finnish variant late-infantile Batten's disease (CLN5), early juvenile Batten's disease, Juvenile Batten disease with granular osmiophilic deposits, infantile NCL, late infantile NCL, juvenile NCL, and other atypical variants of Battens disease, Congenital amaurotic idiocy, Neuronal storage associated with osteopetrosis as described by Takahashi et. al, (Pathol Res Pract, 186:697-706, 1990) and Ambler et. al., (Neurology, 33:437-441, 1988), Niemann-Pick disease including the group I variants (including Groups A and B) and the Group II variants (Groups C, D, and the pure visceral form) and also includes juvenile dystonic lipidosis, juvenile dystonic idiocy without amaurosis, atypical cerebral lipidosis, atypical juvenile lipidosis, subacute Niemann-Pick disease, juvenile Niemann-Pick disease, ophthalmoplegic lipidosis, neurovisceral storage disease with vertical supranuclear ophthalmoplegia, Neville-Lake syndrome, Neville's disease, subacute neurovisceral lipidosis, lactosylceramidosis, sea-blue histiocyte disease, syndrome of the sea-blue histiocyte, chronic reticuloendothelial cell storage disease, and Nova Scotian variant of Niemann-Pick disease, leucodystrophies, mucosulphatidoses associated with one or more defects in many different sulphatases including the Austin variant of metachromatic leucodystrophy and multiple sulphatase deficiency, Krabbe's leucodystrophy including Krabbe's globoid cell leucodystrophy, and Johnny McLeod's disease, neuraminidase deficiency including mucolipidosis I, neuraminidase deficiency group A, neuraminidase deficiency group A subtype 1/i (no dysmorphic features), Cherry-red spot myoclonus syndrome, sialidosis type I, Cherry-red spot/myoclonus syndrome, neuraminidase deficiency group A subtype 2/ii (with dysmorphic features: childhood type), lipomucopolysaccharidosis, Goldberg's syndrome, Sialidosis type II, neuraminidase deficiency group A subtype 3/iii (with dysmorphic features: infantile, severe type), neuraminidase deficiency group B (neuraminidase/beta-galactosidase deficiency[galactosidosis]) subtype 1/i (juvenile-adult type with no or mild dysmorphic features), and neuraminidase deficiency group B (neuraminidase/beta-galactosidase deficiency[galactosidosis]) subtype 2/ii (infantile type with severe or mild dysmorphic features), 1 cell disease, pseudo-Hurler polydystrophy, and other disorders involving defects in N-acetylglucosamine-1-phosphotransferase, mucolipidosis IV, Type II glycogenosis, Pompe's disease, generalized glycogenesis, acid maltase deficiency, lysosomal glycogen storage disease, lysosomal glycogen storage disease without acid maltase deficiency, Farber's lipogranulomatosis, acid esterase deficiency, acid lipase deficiency, and cholesteryl ester storage disease, peroxisomal disorders of infancy, disorders of defective peroxisome assembly such as Zellweger's cerebro-hepato-renal syndrome, and dihydroxyacetone-phosphate acyl transferase deficiency, neonatal adrenoleucodystrophy, adrenoleucodystrophy, infantile Refsum's disease, pseudo infantile Refsum's syndrome, hyperpipecolic acidemia, Zellweger-like syndrome, rhizomelic-infantile chondroplasia punctata (classical type), disorders with single enzyme defects including pseudo-Zellweger's syndrome, 3-oxoacyl coenzyme A thiolase dysfunction/deficiency, pseudo-neonatal adrenoleucodystrophy, peroxisomal bifunctional enzyme deficiency, rhizomelic chondroplasia punctata, bifunctional enzyme deficiency, trihydroxycholestanoic acidemia, pipecolic acidemia (isolated), Refsum's disease, atypical Refsum's disease, glutaric aciduria type III, primary hyperoxaluria, acatalasemia, mevalonic aciduria, Conradi-Hunermann syndrome/disease, X-linked chondroplasia punctata, Conradi-Hunermann chondroplasia punctata, Sjögren-Larsson syndrome as well as other disorders with dysfunctions in peroxisomes, Schilder's disease with adrenal insufficiency, classical adrenoleucodystrophy, childhood adrenoleucodystrophy, mild adrenoleucodystrophy, adult adrenoleucodystrophy, adrenomyeloneuropathy (AMN), adolescent adrenoleucodystrophy, adult cerebral adrenoleucodystrophy, Addison only adrenoleucodystrophy, presymptomatic adrenoleucodystrophy, asymptomatic adrenoleucodystrophy, primary hyperoxaluria type I, and alanine:glyoxalate aminotransferase deficiency/dysfunction/mistargetting and other leucodystrophies including Canavan's disease (van Bogaert and Bertrand type of spongy degeneration), infantile Canavan's disease, congenital Canavan's disease, rapidly progressive Canavan's disease, rapidly progressive infantile Canavan's disease, juvenile Canavan's disease, protracted Canavan's disease, Pelizaeus-Merzbacher disease including the variety of genetic defects in myelin proteolipid protein which give rise to the variety of subtypes of Pelizaeus-Merzbacher disease, Alexander's disease, infantile Alexander's disease, childhood Alexander's disease, juvenile Alexander's disease, adult Alexander's disease, and adult onset Alexander's disease. As used herein, the term “leucodystrophies” refers to adrenoleucodystrophy, adrenomyeloleucodystrophy, and metachromatic leucodystrophies (including sulphatide lipidosis, aryl sulphatase deficiency, cerebroside sulphatase deficiency), globoid cell leucodystrophy (including Krabbe's disease, galactosylceramide lipidosis), X-linked adrenoleucodystrophy (Schilder's disease), neonatal adrenoleukodystrophy, mucosulphatidosis (multiple sulphatase deficiency, Austin variant of metachromatic leucodystrophy), and X-linked leucodystrophy).

Neoplasia

The term “neoplasia”, as used herein, refers to those neuronal deficiencies involving a loss of neurons, damaged neurons, or neurons with inadequate or suboptimal function due to tumors of the nervous system including tumors of neuroepithelial tissue (e.g., astrocytic and ependymal tumors, mixed gliomas, tumors of the choroid plexus and neuroepithelial tumors of uncertain origin such as astroblastomas, polar spongioblastomas, and gliomatosis cerebri), neuronal and neuronal-glial tumors, tumors of the pineal region, embryonal tumors (e.g., medulloepitheliomas, ependymoblastomas, neuroblastomas), tumors of peripheral nerves such as schwannomas and neurofibromas, tumors of the meninges, mesenchymal non-meningothelial tumors, germ cell tumors and tumor-like conditions such as cysts, and plasma cell granulomas, paraneoplastic syndromes, optic nerve tumors of the hypothalamus, posterior pituitary and sellar region. As used herein, the term “neoplasia” excludes lymphomas (e.g., primary malignant lymphomas, secondary lymphomas, and Plasma cell tumors) leukemias, myelomas and polycythemia vera as well as nervous system metastases of tumors which do not arise in the nervous system. The term “paraneoplastic syndromes” excludes paraneoplastic syndromes associated with lymphomas (e.g., primary malignant lymphomas, secondary lymphomas, and Plasma cell tumors) leukemias, myelomas and polycythemia vera.

Neurodegenerative disorders

The term “neurodegenerative disorders” refers to those neuronal deficiencies involving a loss of neurons, damaged neurons, or neurons with inadequate or suboptimal function associated with neurodegenerative disorders such as autosomal recessive proximal spinal muscular atrophy, primary subcortical degenerations (such as Parkinson's disease, multiple system atrophy, Huntington's disease, and progressive supranuclear palsy), familiar and spontaneous Alzheimer's disease, and prion diseases. The term “neurodegenerative diseases” excludes muscular dystrophies, multiple sclerosis, and acquired immunodeficiency syndrome (AIDS), as well as Pick's disease, infantile acid maltase deficiency (Pompe's disease). The term “metabolic neuropathy neurodegenerative diseases” excludes leukodystrophies such as metachromatic leucodystrophy and globoid leucodystrophy.

Neuronal disorders associated with aging and senile dementia

The term “neuronal disorder associated with aging and senile dementia” refers to those neuronal deficiencies involving a loss of neurons, damaged neurons, or neurons with inadequate or suboptimal function due to aging or senile dementia such as changes in dendritic trees (e.g., loss of dendritic spines, swellings, varicosities, and distortions of the horizontal branches, progressive swelling of the cell body, loss of basal dendrites, loss of branches of the apical shaft, loss of terminal branches, loss of apical shaft), decreased synaptic density, shrinkage of neurons, increased lipofuscin content, decreased Nissyl substance, decreases in brain volume, periventricular leucaraiosis, leucaraiosis, accumulation of senile plaques (including amyloid plaques, argyrophilic plaques), accumulation of neurotic plaques, accumulation of non-neurotic plaques, and accumulation of neurofibrillary tangles.

Nutritional disorders

The term “nutritional disorders” refers to those neuronal deficiencies involving a loss of neurons, damaged neurons, or neurons with inadequate or suboptimal function associated with nutritional disorders such as chronic protein-calorie malnutrition or malabsorption (e.g., anorexia nervosa, short bowel syndrome as well as malabsorption associated with cystic fibrosis) and vitamin deficiencies (e.g., thiamine, niacin, vitamin B12 or E deficiency). The term “nutritional disorders,” as used herein, excludes lysosomal storage disorders.

Perinatal neuropathologies

The term “perinatal neuropathologies” refers to those neuronal deficiencies involving a loss of neurons, damaged neurons, or neurons with inadequate or suboptimal function which present during the perinatal period, such as neuronal deficiencies associated disorders such as mental retardation, toxic/metabolic damage such as white and/or grey matter lesions resulting from hypoxia/ischemia (e.g., neuronal cell injury and neuronal necrosis), and prenatal exposure to maternal cocaine and the associated vascular-related lesions, neuronal damage resulting from extracorporeal membrane oxygenation (ECMO) and ECMO associated vascular-related lesions, neuronal damage resulting from congenital heart disease and congenital heart disease associated vascular lesions, kemicterus, neuronal damage resulting from cerebral hemorrhage, neuronal damage resulting from infections such as cytomegalovirus (CMV), neonatal meningitis (including organisms such as Group B streptococcus, E. coli, Staphylococcus, Pseudomonas, Klebsiella), infantile meningitis (including organisms such as Haemophilus influenzae, meningococcemia, and pneumococcus), fungal infections (including organisms such as Candida albicans, Mucor, Crytococcus, Coccidioides, and Aspergillus), TORCH infections (including organisms such as Toxoplasma gondii, rubella, cytomegalovirus, varicella zoster, cocksackie A and B, echovirus, poliovirus, Treponemapallidium, and herpes simplex type 1 and 2), trauma, (e.g., birth trauma, subdural hematoma, spinal cord injury), and neuronal deficiencies resulting from sudden infant death syndrome (SIDS), neuronal deficiencies resulting from neoplasia. As used herein, “perinatal neuropathologies”, and particularly storage/metabolic perinatal neuropathologies, excludes Ceroid lipofuscinosis, deficiencies of GM2 gangliosidases, leucodystrophies such as Sanfilipo's disease, and Zellweger's disease. As used herein, “perinatal neuropathologies”, and particularly “TORCH infections resulting in perinatal neuropathologies” excludes infection by human immunodeficiency virus (HIV). In certain embodiments, the term “perinatal neuropathologies” excludes all lysosomal storage disorders.

Radiation damage

The term “radiation damage”, as used herein, refers to those neuronal disorders involving a loss of neurons, damaged neurons, or neurons with inadequate or suboptimal function associated with X-irradiation induced damage (including acute, early delayed, and late delayed reactions), radiation induced-lesions of the white matter of the brain, the spinal cord, and/or the peripheral nerves. As used herein, “radiation damage” refers to neuronal deficiencies caused by at least 20 Gy for individuals of more than about 2 years of age, at least about 3 Gy for individuals newborn through 2 years of age, and at least about 1 Gy for individuals prenataly exposed to X-radiation.

Schizophrenia

The term “schizophrenia, as used herein, refers to a neuronal deficiency due to a schizophrenic disorder, including disorganized schizophrenia (DSM-IV 295.1), hebephrenic schizophrenia, paranoid schizophrenia (DSM-IV 295.3), residual schizophrenia (DSM-IV 295.6), catatonic schizophrenia (DSM-IV 295.2), simple schizophrenia, simple deteriorative disorder, undifferentiated type schizophrenia (DSM-IV 295.9), and schizophrenia associated with specific syndrome complexes including 1) hallucinations and delusions, 2) disorganized behavior including positive formal thought disorder, bizarre behavior and inappropriate affect, 3) primary, enduring or deficit symptoms, including restricted affective experience and expression, diminished drive, and poverty of thought.

Single gene disorders

As used herein, the term “single gene disorder” refers to a neuronal deficiency due to a defect in a single gene, including Aicardi's syndrome, Angelman's syndrome, Aniridia/Wilm's association, Apert's syndrome, Holoprosencephaly 1, Holoprosencephaly 2, Holoprosencephaly 3, Kallmann's syndrome, Meckel-Gruber syndrome, Miller-Dieker syndrome, Neu-Laxova syndrome, Pallister-Hall syndrome, Pettigrew's syndrome, Prader-Willi syndrome, Sacral agenesis (Currarino triad), Tuberous sclerosis, Waardenburg syndrome type I, Warburg's syndrome, and X-linked hydrocephalus. The term “single gene disorder”, as used herein, excludes lysosomal storage disorders.

Toxic disorders

The term “toxic disorders”, as used herein, refers to neuronal deficiencies involving a loss of neurons, damaged neurons, or neurons with inadequate or suboptimal function associated with exposure to toxins such as metallic toxins including aluminum, arsenic (organic and inorganic), bismuth, cadmium, lead (inorganic and organic), manganese, mercury (inorganic and organic), alkyl mercury compounds, methyl mercury, platinum, tellurium, thallium, tin, alkyl tin compounds, triethy tin, trimethyl tin, as well as syndromes associated with metallic intoxications such as chronic aluminum-induced motor neuron degeneration, dialysis encephalopathy, and human manganism, environmental toxins including acrylamide, acrylamide monomer, carbon disulphide, L-tryptophan, alcohol, ethyl alcohol, ethanol, methanol, methyl alcohol, methyl ester, hexacarbon solvents, n-hexane, methyl-n-butyl ketone, 2,5-hexanedione, formaldehyde, MPTP (N-methyl-4-phenyl- 1,2,3,6-tetrahydropyridine), MPP+ (1-methyl-4-phenylpyridinium), organophosphorus compounds, toluene, styrene, trichloroethylene, xylene, and other solvents, rapeseed oil, oleyl-anilide, as well as syndromes associated with these intoxications such as eosinophilia-myalgia syndrome, fetal alcohol syndrome, “glue sniffing” syndrome, Parkinsonian-like syndrome, organophosphate toxicity syndromes, solvent abuse encephalopathy, and toxic oil syndrome, drug toxicities due to drugs such as Amiodarone, Chloroquine, Clioquinol, Colchicine, Dapsone, Disulfiram (ANTABUSE®), Hexachlorophene (PhisoHex), Isoniazid, Isonicotinic acid hydrazide, Mevacor (LOVASTATIN®), Nitroimidazoles (metronidazole, misonidazole), Perhexiline maleate, Phenytoin (DILANTIN®), Pyridoxine, naturally-occurring (biological) toxic compounds including Buckthom toxin, Cycad (seeds contain cycasin and beta-N-methylamino-L-alanine), Lathyrus sativus (leads to lathyrism, neurolathyrism), 3-Nitropropionic acid (ingestion of fungus Arthinium), Domoic acid, and Psychosine. The term “toxic disorders”, as used herein, excludes neuronal deficiencies associated with treatment of HIV infection (e.g., treatment with ZIDOVUDINE®) or treatment with methotrexate, vincristine, or paclitaxel (TAXOL®). In some embodiments, the term “toxic disorders” excludes neuronal deficiencies associated with administration of amphotericin B.

Trauma

The term “trauma” refers to neuronal deficiencies involving a loss of neurons, damaged neurons, or neurons with inadequate or suboptimal function associated with trauma such as blunt (non-missile) trauma and including focal injury (which includes contusions, intracranial hemorrhage, hematoma, subdural hygroma, tissue tear hemorrhages associated with diffuse axonal injury, and intraventricular hemorrhage), traumatic separation, cranial nerve injury, and injury to blood vessels in the CNS and servicing the CNS, and fat embolism(s)), diffuse injury (which includes diffuse axonal injury, hypoxic (ischemic) brain damage (including that associated with infarction, episodes of hypemia, raised ICP, transient failures of cerebral perfusion pressure, hypotension, cardiac arrest status epilepticus and hypoglycemia), diffuse brain swelling (including that either around focal injuries, or in one or both hemispheres), diffuse vascular injury, multiple small hemorrhages (e.g., petechial hemorrhages) (including that associated with hematological complications associated with thrombocytopenia, small blood vessel disease (often due to sepsis) and adverse drug reactions), dementia pugilistica (punch-drunk syndrome), injury resulting in focal or diffuse (multi-focal) brain damage (including adverse outcomes such as severe neurological disabilities, vegetative state, post-traumatic epilepsy, and progressive neurological disease)), missile head injury, and injury associated with neurosurgery, other surgery, or biopsy. As used herein, the term “trauma” excludes hypoxia related to lymphoreticular proliferative disorders (including lymphoma, leukemia, myeloma, and polycythemia vera).

Vascular disease

The term “vascular disease”, as used herein, refers to neuronal deficiencies involving a loss of neurons, damaged neurons, or neurons with inadequate or suboptimal function associated with vascular diseases including diseases of blood vessels (including stroke, atherosclerosis, hypertensive angiopathy, inflammatory diseases including non-infectious vasculitides, and infectious vasculitides including bacterial vasculitis, granulomatous, and viral vasculitides, aneurysms, vascular malformations, arterial spasm, vascular dementia, and cerebral amyloid angiopathies. -The term “vascular disease” also includes hematologic disorders which result in blood flow abnormalities (including thrombosis, thrombophilia, hyperviscosity, and platelet abnormalities). As used herein, the term vascular diseases excludes systemic lupus erythematosus, thrombophilia associated with antiphospholipid antibodies or polycythemia, anemias (including Sickle cell disease, beta-Thalassemia major, and other thalassemias), lymphoreticular proliferative disorders (including lymphoma, leukemia, myeloma, and polycythemia vera), and viral vasculitides associated with HIV infection. The term “cerebral amyloid neuropathies” excludes amyloidosis associated with myeloma and Waldenstrom's macroglobulinemia.

Psychiatric disorders other than schizophrenia

The term “psychiatric disorders other than schizophrenia”, as used herein, refers to psychiatric disorders including dementia (DSM-IV 290, 290.1, 290.1, 290.11, 290.12, 290.13, 290.2, 290.21, 290.3, 290.4, 290.41, 290.42, 290.43, 294.1, 294.1, 294.1, 294.1, 294.8, 294.8) alcohol induced disorders (DSM-IV 291.1, 291.2, 291.81 291.9), substance abuse-related psychiatric disorders (DSM-IV 292, 292.11, 292.12, 292.81-.84, 292.89, 292.9), psychiatric disorders secondary to a medical condition (DSM-IV 293.83, 293.89, 293.9, 294), cognitive disorders (DSM-IV 294.9), depressive disorders (DSM-IV 296.3, 296.31-.35, 311), bipolar disorders (DSM-IV 296.4, 296.41-.46, 296.5, 296.51-.56, 296.6, 296.61-.66, 296.7, 296.8, 296.89), mood disorders (DSM-IV 296.9), psychotic disorders (DSM-IV 298.9), autistism (DSM-IV 299), narcissistic personality disorder (DSM-IV 301.81), tic disorders (DSM-WV 307.2, 307.22), Tourette's disorder (DSM-IV 307.23), pain disorders (DSM-IV 307.8, 307.89) posttraumatic stress disorder (DSM-IV 309.81), mental retardation, (DSM-IV 317, 318, 318.1, 318.2, 319), neuroleptic-induced Parkinsonism (DMS-IV 332.1), narcolepsy (DSM-IV 347), age-related cognitive decline (DSM-WV 780.9), borderline intellectual functioning (DSM-IV V62.89). The term “psychiatric disorders other than schizophrenia”, as used herein, specifically excludes dementia due to a lysosomal storage disorder (e.g., DSM-IV 290.1) or HIV infection (DSM-IV 294.9).

As used herein, the term “ablative regimen” refers to a treatment protocol or regimen which reduces and/or eliminated circulating white cells, hematopoietic stem cells, and/or hematopoietic precursor cells. Ablative regimens are well known in the art, and generally involve the administration of gamma irradiation and/or cytotoxic chemotherapy.

As used herein, the term “neuronal factors” refers to factors which affect the proliferation, differentiation and/or survival of neurons. Neuronal factors include growth factors, neurotransmitters and the like, as long as they have the biological activity of affecting the proliferation, differentiation and/or survival of neurons.

As used herein, the term “comprising” and its cognates are used in their inclusive sense (i.e., synonymously with “including” and its cognates).

As used herein, the singular form “a”, “an”, and “the” includes plural references unless indicated otherwise. For example, “a” bone marrow-derived cell includes one or more bone marrow-derived cells.

It should be noted that the inventors have disclosed herein a number of disorders involving neuronal deficiencies which have a number of variants and subtypes and which may be referred to by different names by those of skill in the art. The inventors contemplate the inclusion of all subtypes and variants of the neuronal deficiencies disclosed herein, even if the particular subtype or variant is not specifically disclosed. Similarly, this disclosure encompasses all synonyms, eponyms, equivalent terms and/or translations of a particular disorder/syndrome/disease, even if the synonyms, equivalent terms, and/or translations are not specifically disclosed herein. Additional synonyms, eponyms, equivalent terms and translations of neuronal deficiency names, as well as variants and subtypes may be found in GREEFIELD's NEUROPATHOLOGY, (Graham et al., eds., 6th ed., 1997, Oxford University Press, N.Y.) and KAPLAN AND SADOCK's COMPREHENSIVE TEXTBOOK OF PSYCHIATRY (Sadock et al., eds., 7th ed., 2000, Lippincott Williams and Wilkins).

The practice of the present invention will employ, unless otherwise indicated, conventional techniques of molecular biology, microbiology, cell biology, recombinant DNA, and medicine, which are within the skill of the art. See, e.g., Sambrook et al., MOLECULAR CLONING: A LABORATORY MANUAL, 2nd edition (1989); CURRENT PROTOCOLS IN MOLECULAR BIOLOGY, (F. M. Ausubel et al. eds., 1987); the series METHODS IN ENZYMOLOGY (Academic Press, Inc.); PCR 2: A PRACTICAL APPROACH (M. J. McPherson, B. D. Hames and G. R. Taylor eds., 1995); ANIMAL CELL CULTURE (R. I. Freshney. Ed., 1987); and ANTIBODIES: A LABORATORY MANUAL (Harlow et al. eds., 1987).

Bone marrow-derived cells are administered to a subject in need of augmentation of central or peripheral nervous system neurons. The subject, may suffer from a neuronal deficiency, or may otherwise be in need of neuronal augmentation, such as for improvement of memory. The bone marrow-derived cells may be autologous (i.e., derived from the same individual) or syngeneic (i.e., derived from a genetically identical individual, such as a syngeneic littermate or an identical twin), although allogeneic bone marrow-derived cells (i.e., cells derived from a genetically different individual of the same species) are also contemplated. Although less preferred, xenogeneic (i.e., derived from a different species than the recipient) bone marrow-derived cells, such as bone marrow-derived cells from transgenic pigs, may also be administered. When the donor bone marrow-derived cells are xenogeneic, it is preferred that the cells are obtained from an individual of a species within the same order, more preferably the same superfamily or family (e.g., when the recipient is a human, it is preferred that the donor bone marrow-derived cells are derived from a primate, more preferably a member of the superfamily Hominoidea).

Bone marrow-derived cells may be obtained from any stage of development of the donor individual, including prenatal (e.g., embryonic or fetal), infant (e.g., from birth to approximately three years of age in humans), child (e.g., from about three years of age to about 13 years of age in humans), adolescent (e.g., from about 13 years of age to about 18 years of age in humans), young adult (e.g., from about 18 years of age to about 35 years of age in humans), adult (from about 35 years of age to about 55 years of age in humans) or elderly (e.g. from about 55 years and beyond of age in humans).

In some embodiments, the bone marrow-derived cells are administered as unfractionated bone marrow. It is preferred, however, particularly for allogeneic or xenogeneic transplants that the bone marrow be fractionated to enrich for the bone marrow-derived cells prior to administration. Methods of fractionation are well known in the art, and generally involve both positive selection (i.e., retention of cells based on a particular property) and negative selection (i.e., elimination of cells based on a particular property). As will be apparent to one of skill in the art, the particular properties (e.g., surface markers) that are used for positive and negative selection will depend on the species of the donor bone marrow-derived cells.

Methods for fractionation and enrichment of bone marrow-derived cells are best characterized for human and mouse cells, but those of ordinary skill in the art can select homologous markers and methods for fractionating and enriching bone marrow-derived cells from other species.

When the donor bone marrow-derived cells are human, there are a variety of methods for fractionating bone marrow and enriching bone marrow-derived cells known in the art. Positive selection methods such as enriching for cells expressing CD34, and Thy-1 may be used, and negative selection methods which remove or reduce cells expressing CD3, CD10, CD11b, CD14, CD16, CD15, CD16, CD19, CD20, CD32, CD45, CD45R/B220, Ly6G, TER-119 may also be used alone or in combination with positive selection techniques. When the donor bone marrow-derived cells are not autologous, it is preferred that negative selection be performed on the cell preparation to reduce or eliminate differentiated T cells, thereby reducing the risk of graft versus host disease (GVHD).

Generally, methods used for selection/enrichment of bone marrow-derived cells will utilize immunoaffinity technology, although density centrifugation methods are also useful. Immunoaffinity technology may take a variety of forms, as is well known in the art, but generally utilizes an antibody or antibody derivative in combination with some type of segregation technology. The segregation technology generally results in physical segregation of cells bound by the antibody and cells not bound by the antibody, although in some instances the segregation technology which kills the cells bound by the antibody may be used for negative selection.

Any suitable immunoaffinity technology may be utilized for selection/enrichment of bone marrow-derived cells, including fluorescence-activated cell sorting (FACS), panning, immunomagnetic separation, immunoaffinity chromatography, antibody-mediated complement fixation, immunotoxin, density gradient segregation, and the like. After processing in the immunoaffinity process, the desired cells (the cells bound by the immunoaffinity reagent in the case of positive selection, and cells not bound by the immunoaffinity reagent in the case of negative selection) are collected and either subjected to further rounds of immunoaffinity selection/enrichment, or reserved for administration to the patient.

Immunoaffinity selection/enrichment is typically carried out by incubating a preparation of cells comprising bone marrow-derived cells with an antibody or antibody-derived affinity reagent (e.g., an antibody specific for a given surface marker), then utilizing the bound affinity reagent to select either for or against the cells to which the antibody is bound. The selection process generally involves a physical separation, such as can be accomplished by directing droplets containing single cells into different containers depending on the presence or absence of bound affinity reagent (FACS), by utilizing an antibody bound (directly or indirectly) to a solid phase substrate (panning, immunoaffmity chromatography), or by utilizing a magnetic field to collect the cells which are bound to magnetic particles via the affinity reagent (immunomagnetic separation). Alternately, undesirable cells may be eliminated from the bone marrow-derived cell preparation using an affinity reagent which directs a cytotoxic insult to the cells bound by the affinity reagent. The cytotoxic insult may be activated by the affinity reagent (e.g., complement fixation), or may be localized to the target cells by the affinity reagent (e.g., immunotoxin, such as ricin B chain).

It is preferred that bone marrow-derived cells are collected and processed using sterile instruments and techniques, to avoid infectious complications in the recipient. Such techniques are well known in the art. The bone marrow-derived cells administered to the subject may be, or may not be, genetically engineered to produce one or more biological substances of interest, such as a neuronal factor or neurotransmitter. Genetically modified bone marrow-derived cells are utilized when augmentation of the properties of neurons derived from the bone marrow-derived cells is desired or when the production of secreted factor(s) (e.g., a neurotrophic or gliotrophic factor) in the CNS or PNS is desirable, although in certain embodiments, such as Parkinson's disease (and its subtypes) and Parkinsonism, the use of bone marrow-derived cells which have not been genetically modified to produce L-DOPA or dopamine is contemplated. Generally, a construct encoding a molecule (often an enzyme or structural protein) that is desirable in the disorder to be treated is introduced into the bone marrow-derived cells. The construct may employ a ubiquitous promoter (beta-actin, for example), but neuron-specific promoters, such as the promoters for NeuN (neuronal nuclei), Calmodulin-dependent Protein Kinase II (CaMKII), Calmodulin-dependent Protein Kinase IV (CaMKIV), any of the neurofilaments (including the 200 kD, 160 kD, 150 kD, 145 kD, 70 kD, and 65 kD forms), class III beta-tubulin calbindin D-28k, microtubule associated protein 2, synaptic protein SNAP-25, synaptophysin, NMDA receptor, neuron specific enolase, tyrosine hydroxylase, neural nestin, synapsin-1, tau, Hu, doublecortin, and the like, are preferred. For example, when the bone marrow-derived cells are utilized for the treatment of Parkinson's disease, the cells may be modified to express the enzymes necessary for dopamine production (e.g., tyrosine hydroxylase; Wolff et al., 1989, Proc. Natl. Acad. Sci. USA 86(22):9011-9014, and/or L-DOPA decarboxylase, Scherer et al., 1992, Genomics 13(2), 469-471). See, for example Gage et al. (1987, Neuroscience 23:795-807). Other examples include differentiation of marrow-derived cells into GABA-containing neurons in the basal ganglia to replace those lost in patients with Huntington's disease, and production of cholecystokinine by marrow-derived cells implanted into the temporal cortex or hippocampus to treat schizophrenia.

Introduction of genetic constructs into bone marrow-derived cells can be accomplished using any technology known in the art, including calcium phosphate-mediated transfection, electroporation, lipid-mediated transfection, naked DNA incorporation, electrotransfer, and viral (both DNA virus and retrovirus mediated) transfection. Methods for accomplishing introduction of genes into cells are well known in the art (see, for example, Ausubel, id.).

As will be apparent to one of skill in the art, it may be desirable to subject the recipient to an ablative regimen prior to administration of the bone marrow-derived cells. Ablative regimens typically involve the use of gamma radiation and/or cytotoxic chemotherapy to reduce or eliminate endogenous hematopoietic cells, such as circulating white cells and/or hematopoietic stem cells and precursors. A wide variety of ablative regimens using chemotherapeutic agents are known in the art, including the use of cyclophosphamide as a single agent (50 mg/kg q day×4), cyclophosphamide plus busulfan, the DACE protocol (4 mg decadron, 750 mg/m² Ara-C, 50 mg/m² carboplatin, 50 mg/m² etoposide, q 12 h ×4 IV), and the like. Additionally, gamma radiation may be used (0.8 to 1.5 kGy, midline doses) alone or in combination with chemotherapeutic agents. In accordance with standard practice in the art, when chemotherapeutic agents are administered, it is preferred that the be administered via an intravenous catheter or central venous catheter to avoid adverse affects at the injection site(s).

The bone marrow-derived cells are administered to a subject having a neuronal deficiency. Those of skill in the art (i.e., medicine, surgery, and psychiatry) will recognize subjects having neuron deficiencies, as described herein, using techniques known in the art.

Neuronal deficiency may include loss of a memory function such as, amnesia. Amnesia is an inability to recall information that is stored in the memory. There are three types of memory affected by amnesia including immediate memory, intermediate memory and long term memory. When the immediate memory is affected the patient has difficulty recalling the events that occurred in the preceding few seconds. Intermediate memory is affected when the patient cannot recall events that happened from within a few seconds to a few days prior to the cause of the amnesia. With long term memory loss the patient will be unable to recall events that occurred further back in time. Examples of memory functions include “episodic” memory (memory for events) and “semantic” memory (memory for facts) which can be lost when the memory system is damaged. Memory functions may also be classified as sensory memory, short-term memory and long-term memory.

Bone marrow-derived cells are preferably formulated in a physiologically acceptable solution (e.g., normal saline, buffered saline, or a balanced salt solution) and administered to the subject by vascular administration (e.g., intravenous infusion), in accordance with art accepted methods utilized for bone marrow transplantation. Typically, an infusion catheter is inserted into a vein, and a single cell suspension of bone marrow-derived cells is infused into the recipient subject. Preferably, the bone marrow-derived cells are administered into a peripheral vein, more preferably a superficial peripheral vein, but central venous administration (e.g., through a central venous catheter) is also contemplated. Additionally, cells may be administered by direct injection into the CNS (brain or spinal cord) or by intrathecal injection or infusion, although these routes are less preferred. It is preferred that the catheter or needle used for administration be relatively large gauge (e.g., larger than about 20 gauge) to avoid blockage of the catheter or needle by any clumps of sells present in the bone marrow-derived cell preparation.

An effective amount of bone marrow-derived cells are administered to the recipient. Preferably, at least about 10₂ and less than about 10₉ cells are administered to the recipient. The number of bone marrow-derived cells administered may range from about 10¹, 10², 5 ×10², 10³, 5×10³, 10⁴, 10⁵, 10⁶, 10⁷, or 10⁸ to about 5×10², 10³, 5×10³, 10⁴, 10⁵, 10⁶, 10⁷, 10⁸, or 10⁹, where the upper and lower limits are selected independently, except that the lower limit is always less than the upper limit. The number of cells administered will depend on both the neuronal deficiency to be treated as well as the level of fractionation of the bone marrow-derived cells. As will be apparent to one of skill in the art, the number of cells necessary to form an “effective amount” decrease as the degree of fractionation or purity increases.

The bone marrow-derived cells may be delivered in a single administration or in multiple (i.e., greater than one) administrations. When the bone marrow-derived cells are delivered in multiple administrations, the spacing of the multiple administrations may be uniform or varying, but the various administrations are preferably at least one day apart, and may be separated by at least 2, 3, 4, 5, 7, 9, 11, 14, 21, 28, or more days.

In some instances, bone marrow-derived cells are administered in conjunction with one or more neuronal factors affecting the proliferation, differentiation and/or survival of neurons. Neuronal factors are well known in the art, and include nerve growth factor (NGF), brain-derived neurotrophic factor (BDNF), neurotrophin-3, -4,-5, -4/5 and -6 (NT-3, -4,-5, -4/5, -6), ciliary neurotrophic factor (CNTF), glial-derived neurotrophic factor (GDNF), growth promoting activity (GPA), luteinizing hormone releasing hormone (LHRH), KAL gene (implicated in X-linked Kallman's syndrome), insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF- 1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-β1, -β2, -β3, -β4, and -β5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies). As will apparent to those of skill in the art, biologically active fragments and peptide mimetics may be used in addition to or instead of full length neuronal factors. Additionally, the bone marrow derived stem cells may be genetically engineered (e.g., by any DNA transformation, viral transduction, or any other genetic transduction technique known in the art) to produce the neuronal factor(s) themselves.

In certain embodiments, the neuronal factor(s) are produced by additional cells. Cells which endogenously produce a neuronal factor or which have been genetically modified to produce a neuronal factor may be used. The additional cells may be mixed with the bone marrow-derived stem cells prior to or at the time of administration, or they may be administered separately.

Additionally or alternatively, bone marrow-derived cells may be engineered to express receptors for neuronal factor(s) such as trkA, trkA[EI] (extracellular 6-amino-acid insert), trkb, trkB[T1], trkB[T2], trkC, trkC[TK+14] (14-amino-acid kinase insert), trkC[TK+25] (25-amino-acid kinase insert), trkC[TK+39] (39-amino-acid kinase insert), trkC[TK-158] (158-amino-acid deletion), trkC[TK-143] (143-amino-acid deletion), trkC[TK-113] (113-amino-acid deletion), trkC[TK-108] (108-amino-acid deletion), or p75-LNTR (low-affinity neurotrophin receptor or low-affinity NGF receptor).

The neuronal factor may be admixed with the bone marrow-derived cells or administered separately. When the neuronal factor is administered separately from the bone marrow-derived cells, the neuronal factor may be administered systemically (e.g., by parenteral administration, such as IV, subcutaneous, intramuscular, or intraperitoneal), but is preferably administered to the nervous system (e.g., by direct injection into the brain or spinal cord or by intrathecal injection/infusion). Administration of the neuronal factor may be by bolus or by infusion.

Alternately, or in addition to the administration of a neuronal factor, tissue damage may also be used to induce the endogenous production of neuronal factors at either the target site or a different site. Tissue damage may be produced by any means convenient, most commonly by directly creating mild physical damage at sites in the nervous system using a probe, needle, catheter, or the like.

Administration of bone marrow-derived cells to a subject results in the formation of new neurons, derived from the bone marrow-derived cells, in the nervous system of the patient. Administration of bone marrow-derived cells results in an improvement, stabilization, or a reduction in the rate of progression of symptoms of a neuronal deficiency. The symptoms of neuronal deficiencies are well known in the art, as are methods of assessing the severity of symptoms. As will be understood by one of skill in the art, the exact symptoms will depend on the disorder and the particular patient, as neuronal deficiencies are generally pleiomorphic and follow varying natural histories in different individuals.

The invention also provides for treatment of a neuronal deficiency by administration of a bone marrow cell mobilization treatment. Bone marrow cell mobilization protocols are well known in the art. The use of granulocyte colony stimulating factor (G-CSF) for bone marrow cell mobilization is well known (see, e.g., Chao et al., 1993, Blood 81(8):2031-2035). The recombinant form of human G-CSF is commercially available as filgastrim. Recombinant human GM-CSF is also commercially available and is used in bone marrow cell mobilization protocols. Commonly used protocols involve the administration of 5-24 μg/kg/day of G-CSF, preferably about 10 to 12 μg/kg/day, for four, five or six days. GM-CSF may also be used alone, but is more preferably used in combination with G-CSF, for example in a protocol administering about 10 μg/kg/day of G-CSF with 5 μg/kg/day GM-CSF for four, five or six days (Körbling, 1999, Bailliéres Clin. Haem. 12(1/2):41-55).

G-CSF and/or GM-CSF may also be combined with additional agents. Flt-3 ligand (from about 1 to about 100 μg/kg/day) may be combined with G-CSF and/or GM-CSF. U.S. Pat. No. 5,925,568 discloses the use of MIPα for bone marrow cell mobilization. Additionally, the use of anti-VLA-4 antibody and/or an anti-VCAM-1 is disclosed in U.S. Pat. No. 5,843,438.

Additionally, a bone marrow cell mobilization therapy may be combined with the administration of bone marrow-derived stem cells for treatment of any of the disorders for which treatment by administration of bone marrow-derived stem cells is disclosed herein.

In many instances, generalized end points may be used to assess symptoms of neuron deficiencies. For example, activities of daily living (ADLs) are useful end points for assessing the integration of physical and mental function. ADLs include moving to and from bed, walking, sitting in and rising from a chair, bathing, dressing, cooking, feeding, and the like. Tests of mental function may also be of use, including the Monumental Status Examination, the Iowa Battery for the Detection of Mental Decline, the Wechsler Adult Intelligence Scale, the Wechsler Memory Scale, the Benton Visual Retention Test, the Stanford-Binet intelligence quotient examination, and the like. Additional tests useful in assessing symptoms of neuronal deficiencies include tests of directed movement, reaction time, grip and limb strength, Babinski sign, and the like. Radiological imaging may also be helpful; for example, computerized tomography (CT) and magnetic resonance imaging (MRI) scans are useful for assessing lesion number and size, and positron emission tomography (PET) scans may be used to assess functionality of particular portions of the nervous system. Combinations of selected assays are useful for particular disorders. For example, tests such as ADLs, directed movement and reaction time are useful in assessment of PD, ADLs, grip and limb strength, and Babinski's sign are useful in assessment of ALS, and ADLs, grip and limb strength, and CT or MRI scanning are useful in assessment of MS. Electrophysiological methods such as tests of nerve conduction, electroencephalograms, and the like may be used to assay nerve function. Additionally, clinimetric scales are also useful for assessment of the symptoms of neuronal deficiency disorders. Clinimetric scales are useful for quantification of overall health (e.g., Karnofsky performance score) and for symptoms of specific disorders. For example, the Chalfont Seizure Severity Scale and the Liverpool Seizure Severity Scale (Duncan et al., 1991, J. Neurol. Neurosurg. Psychiatry 54:873-876; Baker et al., 1991, Epilepsy Res. 8:245-251) are useful for measurement of epilepsy symptoms, while the Pourcher and Barbeau ataxia rating scale (1980, Can. J Neurol. Sci. 7:339-344) is useful in assessing symptoms of ataxia.

### EXAMPLES

Example 1: Identification of bone marrow-derived cells in the CNS

Bone marrow-derived cells were sterilely harvested from C57B/6 mice which had been modified to produce green fluorescent protein (GFP) in every cell. Marrow was harvested by flushing 2% fetal calf serum (FCS) in Hank's buffered salt solution (HBSS) through the marrow cavities of the limb bones with a 25 gauge needle. Cells were collected and suspended in 2% FCS in HBSS, filtered through 70 μm NITEX® (Tetko, Inc.) mesh, collected by centrifugation (approximately 400×g for 5 minutes), and then resuspended at 4.8×10⁷ nucleated cells/mL.

Isogeneic recipient mice were prepared for transplant by lethal irradiation (950 cGy total dose, split into equal fractions and administered 3 hours apart). The bone marrow-derived cells were administered by injection of 125 μL of the cell suspension into the tail vein.

Approximately 3 months after transplant, recipient mice were euthanized by cervical dislocation and brain cells were isolated from the recipient mice by opening the cranium, removing the brain, mincing the brains with a razor blade, rinsing the minced tissue twice with HBSS, and resuspending in 10 mL of PPD solution (2.5 U/mL papain, 250 u/mL DNase I, 1 u/mL dispase II, in HBSS plus 12.4 mM MgSO₄). The tissue was incubated at 37° C. for 30 minutes, then digestion was stopped by the addition of 2 mL of fetal bovine serum (FBS). The tissue was dissociated by trituration, then filtered through a 70 mm sieve (BD Biosciences) and washed 3 times with 20% FCS in Dulbecco's Modified Eagles medium (DME).

Cells were resuspended in 200 mL of 5% FBS in phosphate buffered saline (PBS) and incubated on ice for 15 minutes with Tricolor (TC)-conjugated rat anti-mouse CD 11b and Allophycocyanin (APC)-conjugated rat anti-mouse CD45. Control cells were incubated with isotype matched TC- and APC- conjugated specific for irrelevant antigens. The cells were washed once with 5% FBS in PBS (FBS/PBS), and resuspended in 200 mL of FBS/PBS and fixed by addition of 400 mL of solution A from the “Fix and Perm” kit (Caltag) and incubation at room temperature for 15 minutes. The cells were washed twice, then stained for nuclear DNA by incubation in FBS/PBS containing 0.12 mg/mL Hoechst 33258. Cells were analyzed using a MOFLO® flow cytometer (Cytomation, Inc.) and FLOJO® software (Tree Star, Inc.).

A distinct population of GFP+ cells was identified in animals transplanted with GFP+ bone marrow, as compared to cells from animals transplanted with non-GFP expressing marrow. Approximately 95% of the GFP+ cells were positive for CD11b and/or CD45, markers of myelomonocytic cells and circulating white cells, respectively. Approximately 5% of the GFP+ cells were clearly negative for both hematopoietic cell markers.

In a similar experiment, GFP+ cells from dissociated brain were stained with antibodies recognizing Hu, which is a nuclear protein only expressed in neurons, and Hoechst 33258, which stains DNA. The anti-Hu antibody was detected with a secondary antibody labeled conjugated to Texas Red. The stained cells were embedded in a collagen matrix and evaluated by epifluorescence microscopy which revealed that 3% of GFP+ cells expressed the neuronal protein Hu. Further analysis confirmed that the anti-Hu staining was localized to the nuclear regions of isolated GFP-positive, bone marrow-derived cells. These finding suggested that exposure to the CNS environment may have led a subpopulation of bone marrow-derived cells to acquire a novel neuronal phenotype.

Example 2: Identification of bone marrow-derived neurons

Animals were prepared and transplanted with GFP+ bone marrow-derived cells as described in Example 1. 8 to 12 weeks after transplant, the recipient mice were euthanized and perfused with 25 mL of 4° C. phosphate buffer (pH 7.4) followed by 25 mL of 4° C. 1.5% paraformaldehyde in phosphate buffer. Brains were removed and incubated in 1.5% paraformaldehyde, 0.1% glutaraldehyde, 20% sucrose in phosphate buffer overnight at 4° C. The brains were embedded in TISSUE-TEK® O.C.T. compound (Sakura Finetek) and snap frozen. 20-40 μm coronal cryosections were taken from the olfactory bulb (Bregma −4.1 to −3.6).

Sections were blocked with 25% normal goat serum (NGS), 0.25% Triton® X-100, and rat anti-mouse-CD16/32 (1:1000, Pharmingen) in PBS for one hour. The sections were stained with anti-NeuN (MAB377 from Chemicon, 1:4000), anti-200 kD neurofilament (AB1989 from Chemicon, 1:400), anti-beta3-microtubulin (TUJ1 from Covance, 1:1000) anti-glial fibrillar acid protein (GFAP; polyclonal antibody, Dako, 1:2000) or anti-F4/80 (Caltag, 1:800) antibodies for 48 hours at 4° C., washed, then incubated with the appropriate secondary antibody (Goat anti-mouse and goat anti-rabbit antibodies conjugated to Texas Red or Cy5, 1:800, Molecular Probes, Inc.). The sections were imaging using a laser confocal microscope adjusted to yield optical sections with a theoretical thickness of 0.3 to 0.4 μm. Sequential laser excitation was employed to eliminate bleedthrough.

An average of 220 (SD+ 96) GFP+ cells were observed per section of the olfactory bulb (OB). Of these GFP+ cells, the majority, 72%, expressed the F4/80 microglial surface marker. Many of the GFP+/F4/80− cells had morphologies suggestive of neuronal cells.

The morphology of GFP+ cells was analyzed by visual inspection using epifluorescence and laser scanning confocal microscopy. The majority of GFP+ cells that co-stained for neuronal markers (NeuN or NF-H) were triangular in morphology (61.7% and 60.9%, respectively), while F4/80+ cells were mostly spindle or stellate in morphology. Because neurons in the CNS often assume triangular morphology, cells with triangular morphology were subdivided into three categories: those having no observable extensions (+), those having a single observable extension of less than 10 μm (++), and those having either a single observable branched extension or more than one observable extension (+++). Results of the morphological analysis are summarized in Table 1.

TABLE 1

| | | Markers | | |
|---|---|---|---|---|
| | | NeuN+ | NF-H+ | F4/80+ |
| Morphology | | (n = 165) | (n = 129) | (n = 229) |
| Triangular | + | 13.3% | 34.6% | 18.0% |
| | ++ | 34.4% | 18.9% | 3.5% |
| | +++ | 14% | 7.4% | 3.5% |
| Round | | 12.3% | 9.4% | 0 |
| Oval | | 8.6% | 10.9% | 3.6% |
| Rod | | 2.5% | 0 | 0 |
| Spindle | | 1.2% | 10.9% | 35.7% |
| Stellate | | 2.5% | 2.3% | 32.1% |
| Other | | 11.1% | 2.4% | 3.6% |

Sections of the OB were also analyzed with respect to localization of bone marrow-derived cells. Coronal sections of the OB were stained for a single marker and analyzed with respect to GFP+ cells in each layer of the OB. 12 sections, averaging 10,400 (±600) neurons per section, were analyzed for localization of GFP+ neurons (8 for NeuN+ cells, 4 for NF-H+ cells), 4 sections, averaging 2000 (±200) astrocytes per section, were analyzed for localization of GFP+ astrocytic cells, and 3 sections, averaging 550 (±50) microglia per section, were analyzed for localization of GFP+ microglial cells. The majority of GFP+ cells were found in the superficial axon layer (SAL), and relatively large numbers of GFP+ cells were also found in the glomerular layer. Interestingly, no GFP+ cells expressing an astrocytic marker (glial acid fibrillar protein, GFAP) were identified in any of the sections, contrary to previously published reports (Eglitis et al., id.). The results of the anatomic analysis of the OB are summarized in Table 2.

TABLE 2

| | GFP+ Cells | | | |
|---|---|---|---|---|
| | Neurons | | Astrocytes | Microglia |
| Layer | NeuN+ | NF-H+ | GFAP+ | F4/80+ |
| Superficial Axon | 105 | 66 | 0 | 312 |
| Glomerular | 30 | 41 | 0 | 114 |
| External | 14 | 10 | 0 | 56 |
| Plexiform | | | | |
| Mitral Cell | 4 | 0 | 0 | 7 |
| Internal | 0 | 0 | 0 | 8 |
| Plexiform | | | | |
| Granule | 12 | 11 | 0 | 13 |
| Total | 165 | 129 | 0 | 510 |

All publications and patent applications mentioned in this specification are incorporated herein by reference to the same extent as if each individual publication or patent application were specifically and individually indicated to be incorporated by reference.

The above description is illustrative and not restrictive. Many variations will be apparent to those skilled in the art upon review of this disclosure. The scope of the invention should not be determined with reference to the above description, but instead should be determined with reference to the appended claims and the full scope of their equivalents.

We claim:

1. A method for treating a neuronal deficiency, comprising:
administering bone marrow-derived cells to an individual having a neuronal deficiency, wherein the administering of the bone marrow-derived cells induces a formation of new neurons in a nervous system of the subject; and
ameliorating at least one symptom of the neuronal deficiency.

2. The method of claim 1, wherein said neuronal deficiency arises from a disorder selected from the group consisting of abnormalities of the central autonomic systems, congenital disorders and disorders arising from teratogen exposure, demyelinating diseases, diseases of peripheral nerves, disorders of the hypothalamus and pituitary, disorders of movement, disorders of the spinal cord and vertebral column, epilepsy, hypoxia, increased intracranial pressure, infectious disease, neoplasia, neurodegenerative disorders, neuronal disorders associated with aging and senile dementia, nutritional disorders, perinatal neuropathologies, radiation damage, schizophrenia, single gene disorders, toxic disorders, trauma, vascular disease, and psychiatric disorders other than schizophrenia.

3. The method of claim 2, wherein said neuronal deficiency is not a neuron deficiency arising from a disorder selected from the group consisting of:
a lysosomal or peroxisomal disorder, Zellweger's disease, human immunodeficiency virus (HIV) infection, multiple sclerosis (MS), adrenoleucodystrophy, adrenomyeloneuropathy, a metachromatic leucodystrophy, a sulphatide lipidosis, globoid cell leucodystrophy, amyotrophic lateral sclerosis, amyotrophic lateral sclerosis with frontal lobe dementia, a bone marrow ablation treatment, lymphoreticular disorders, metastases of tumors which do not arise in the nervous system, infantile acid maltase deficiency (Pompe's disease), Ceroid lipofuscinosis, a deficiency of GM2 gangliosidase, Sanfilippo's disease, leucodystrophy, systemic lupus erythematosus, thrombophilia associated with antiphospholipid antibodies or polycythemia, and anemia including Sickle cell disease, beta-Thalassemia major, and other thalassemias.

4. The method of claim 1, wherein said bone marrow-derived cells are autologous.

5. The method of claim 4, wherein said autologous bone marrow-derived cells are genetically modified.

6. The method of claim 1, wherein said bone marrow-derived cells are allogeneic.

7. The method of claim 6, wherein said allogeneic bone marrow-derived cells are genetically modified.

8. The method of claim 1, wherein said bone marrow-derived cells are administered in conjunction with a neuronal factor.

9. The method of claim 8, wherein said neuronal factor is selected from the group consisting of:
nerve growth factor (NGF), brain-derived neurotrophic factor (BDNF), neurotrophin-3, -4, -5, -4/5 and -6 (NT-3, -4, -5, -4/5, -6), ciliary neurotrophic factor (CNTF), glial-derived neurotrophic factor (GDNF), growth promoting activity (GPA), luteinizing hormone releasing hormone (LHRH), KAL gene, insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF- 1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-β1, -β2, -β3, -β4, and -β5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies).

10. The method of claim 8, wherein said neuronal factor is administered with the bone marrow-derived cells.

11. The method of claim 8, wherein said neuronal factor is administered separately from said bone marrow-derived cells.

12. The method of claim 11, wherein said neuronal factor is administered intrathecally.

13. The method of claim 1, further comprising the step of mildly damaging the nervous system of the individual.

14. The method of claim 1, wherein said bone marrow-derived cells are administered by vascular administration.

15. The method of claim 14, wherein said bone marrow-derived cells are administered by intravenous administration.

16. The method of claim 15, wherein said bone marrow-derived cells are administered by intravenous infusion.

17. The method of claim 16, wherein said intravenous infusion is into a peripheral vein.

18. The method of claim 1, wherein said bone marrow-derived cells are administered intrathecally.

19. The method of claim 1, wherein said bone marrow-derived cells are administered by direct administration into a site in said subject's nervous system.

20. The method of claim 19, wherein said site in the subject's nervous system is in the subject's central nervous system (CNS).

21. The method of claim 1, wherein said subject is a human.

22. A method for improving memory function in an individual with deficient memory function, comprising:
administering bone marrow-derived cells to an individual having deficient memory function, wherein the administering of the bone marrow-derived cells induces a formation of new neurons in a nervous system of the subject; and
improving at least one memory function in said individual.

23. The method of claim 22, wherein said bone marrow-derived cells are autologous.

24. The method of claim 23, wherein said autologous bone marrow-derived cells are genetically modified.

25. The method of claim 22, wherein said bone marrow-derived cells are allogeneic.

26. The method of claim 25, wherein said allogeneic bone marrow-derived cells are genetically modified.

27. The method of claim 22, wherein said bone marrow-derived cells are administered in conjunction with a neuronal factor.

28. The method of claim 27, wherein said neuronal factor is selected from the group consisting of wherein said neuronal factor is selected from the group consisting of nerve growth factor (NGF), brain-derived neurotrophic factor (BDNF), neurotrophin-3, -4, -5, -4/5 and -6 (NT-3, -4, -5, -4/5, -6), ciliary neurotrophic factor (CNTF), glial-derived neurotrophic factor (GDNF), growth promoting activity (GPA), luteinizing hormone releasing hormone (LHRH), KAL gene, insulin, insulin-like growth factor-I-alpha, I-beta, and -II (IGF-I-alpha, I-beta, -II), interleukins (e.g., IL-2, IL-6, and the like), platelet derived growth factors (including homodimers and heterodimers of PDGF A, B, and v-sis), retinoic acid (especially all-trans-retinoic acid), fibroblast growth factors (FGFs, e.g., FGF-1, -2, -3), epidermal growth factor (EGF), leukemia inhibitory factor (LIF), the neuropeptide CGRP, vasoactive intestinal peptide (VIP), gliobastoma-derived T cell suppressor factor (GTSF), transforming growth factor alpha, epidermal growth factor, transforming growth factor betas (including TGF-β1, -β2, -β3, -β4, and -β5), vascular endothelial growth factors (including VEGF-1, -2, -3, -4, and -5), stem cell factor (SCF), neuregulins and neuregulin family members (including neuregulin-1 and heregulin), netrins, galanin, substance P, tyrosine, somatostatin, enkephalin, ephrins, bone morphogenetic protein (BMP) family members (including BMP-1, -2, -3 and -4), semiphorins, glucocorticoids (including dexamethasone), progesterone, putrescine, supplemental serum, extracellular matrix factors (including laminins, fibronectin, collagens, glycoproteins, proteoglycans and lectins), cellular adhesion molecules (including N-CAM, L1, N-cadherin), and neuronal receptor ligands (including receptor agonists, receptor antagonists, peptidomimetic molecules, and antibodies).

29. The method of claim 27, wherein said neuronal factor is administered with the bone marrow-derived cells.

30. The method of claim 27, wherein said neuronal factor is administered separately from said bone marrow-derived cells.

31. The method of claim 30, wherein said neuronal factor is administered intrathecally.

32. The method of claim 22, wherein said memory function is a short term memory function.

33. The method of claim 22, wherein improving said memory function comprises stabilizing said memory function.

34. A method for treating a neuronal deficiency, comprising:
administering a bone marrow cell mobilization therapy to an individual having a neuron deficiency, wherein the administering of the bone marrow cell mobilization therapy induces formation of new neurons in the nervous system of the subject; and
ameliorating at least one symptom of the neuronal deficiency.
